(19) United States
(12) Patent Application Publication
(10) Pub. No.: US 2006/0122522 A1
(43) Pub. Date: Jun. 8, 2006

(54) Devices and methods for positioning and anchoring implantable sensor devices

(76) Inventors: Abhi Chavan, Maple Grove, MN (US); Jeffrey A. Von Arx, Minneapolis, MN (US); Bruce Tockman, Scandia, MN (US); Donald Palme, Princeton, MN (US)

Correspondence Address: JASON R. KRAUS;FAEGRE & BENSON, LLP, 2200 WELLS FARGO CENTER, 90 SOUTH SEVENTH STREET, MINNEAPOLIS, MN, 55402-3901, US

(21) Appl. No.: 11/216,738

(22) Filed: Aug. 31, 2005

Related U.S. Application Data
(60) Provisional application No. 60/633,183, filed on Dec. 3, 2004.

(51) Int. Cl. A61B 5/02

(52) U.S. Cl. 600/505

(57) ABSTRACT

Methods and systems provide for anchoring implantable medical device inside a bodily vessel. An anchoring structure can include a stent-like structure to which the IMD is attached. The stent-like structure is positioned at a desired location in the bodily vessel. The stent-like structure can be repositioned based on a measurement from the IMD. The IMD can include outwardly extending fins over which tissue can fibrose to affix the IMD to a wall of the bodily vessel. The stent-like structure can be made of a bio-absorbable material. The IMD can be attached to a stent-like structure by leads, by being lodged in a recessed diaphragm, by being embedded in mesh of the stent-like structure, or other methods. The stent-like structure can be balloon deployable to allow for controlled positioning and anchoring. The anchoring structure can include a vena cava filter.

### CROSS-REFERENCE TO RELATED APPLICATIONS

This application claims the benefit of U.S. Provisional Application No. 60/633,183 filed on Dec. 3, 2004.

### TECHNICAL FIELD

Embodiments of present invention generally relate to medical devices that can be implanted in a patient's body, and more particularly, to apparatus and methods for delivering, positioning, and anchoring the implantable medical devices (IMDs) within a patient.

### BACKGROUND

Medical devices are known that can be implanted within a patient's body for monitoring one or more physiological parameters and/or to provide therapeutic functions. For example, sensors or transducers can be placed in the body for monitoring a variety of properties, such as temperature, blood pressure, strain, fluid flow, chemical properties, electrical properties, magnetic properties, and the like. In addition, medical devices can be implanted that perform one or more therapeutic functions, such as drug delivery, cardiac pacing, defibrillation, electrical stimulation, and the like.

As mentioned above, such implantable medical devices (IMDs) can be configured to measure or sense a number of different parameters in the body. One parameter of particular interest is blood pressure. Implantable pressure sensing modules used in conjunction with cardiac rhythm management (CRM) devices show promise for being able to predict the onset of pulmonary edema in congestive heart failure patients. In addition, certain pressure sensors also may have applications in monitoring and treating hypertension, in automatic CRM device settings optimization, and in rhythm discrimination.

Obviously, to reap the benefit of an IMD, the IMD must be implanted in the patient's body. Implanting generally involves delivering and anchoring the IMD at a desired location in the body. The delivery and anchoring methods and mechanisms that are used can be critical in determining the effectiveness of the IMD and patient wellbeing. For example, the manner in which the IMD is implanted can be important for patient safety, device placement control, sensor accuracy, long term stability, and physician acceptance and adoption.

With regard to blood pressure, there are a number of locations where the pressure can be measured. For example, as one skilled in the art will appreciate, pressures measured in the pulmonary artery can be reflective of end diastolic pressures on the left side of the heart. Unfortunately, current approaches do not effectively allow for positioning and anchoring IMDs within the pulmonary artery.

Thus, a need exists for apparatus and/or methods for placing and anchoring implantable medical devices within a patient's body.

### SUMMARY

Embodiments are described that provide for anchoring a sensor in a bodily vessel. In some embodiments, an anchoring device includes a stent-like structure adapted to be secured in the bodily vessel. The stent-like structure can include a reservoir integrally formed with the stent-like structure for holding and/or securing a sensor. The sensor may be operable to measure a physiologic parameter value in the bodily vessel. In one embodiment, the physiologic parameter value being measured by the sensor is blood pressure. Further, in some embodiments, the sensor can be operable to communicate the physiologic parameter value to an implantable medical device and/or an external computing device.

In yet another embodiment, the present invention relates to an anchoring device for anchoring a sensor in a bodily vessel, which comprises a stent-like structure adapted to be secured in the bodily vessel, and a sensor/lead assembly. In accordance with this particular embodiment, the sensor/lead assembly comprises a sensor operable to measure a physiologic parameter in the bodily vessel, a first lead portion connecting the sensor to an implantable medical device, and a second lead portion connecting the sensor to the stent-like structure. Thus, in this embodiment, the stent-like structure is operable to anchor the sensor/lead assembly in a desired location within the bodily vessel. In accordance with this particular embodiment, the sensor can communicate with the implantable medical device via the first lead portion, or the sensor can communicate with the implantable medical device via a wireless communication connection.

In yet another embodiment, the present invention relates to an anchoring device for anchoring a sensor in a bodily vessel, which comprises a stent-like structure adapted to be secured in the bodily vessel, and an alternative sensor/lead assembly. In this particular embodiment, the sensor/lead assembly includes a sensor connected to the stent-like structure, and a lead connecting the sensor to an implantable medical device. Thus, in this embodiment, the stent-like structure is operable to anchor the sensor/lead assembly in a desired location within the bodily vessel. Further, in accordance with this particular embodiment, the sensor can communicate with the implantable medical device via the lead, or the sensor can communicate with the implantable medical device via a wireless communication connection.

In yet another embodiment, the present invention relates to an anchoring assembly for anchoring a sensor in a bodily vessel. In this particular embodiment, the anchoring assembly comprises an anchoring device comprising a bio-absorbable material, and a sensor having one or more extensions. As discussed above, the sensor is operable to measure one or more physiologic parameters in the bodily vessel. Further, the sensor is connected to the anchoring device in a location such that at least the extensions of the sensor can be secured near a wall of the bodily vessel. Thus, in this embodiment, when the anchoring device and sensor are placed in the bodily vessel, the extensions of the sensor are located near the wall of the vessel so that vessel tissue can grow over at least a portion of the extensions holding the sensor in place. Then, the anchoring device can absorb or dissolve after the vessel tissue grows over the at least a portion of the extensions, so that only the sensor and extensions are left in the vessel. In one embodiment, the extensions can be formed of the same material as the sensor. In an alternative embodiment, the extensions can be formed of a bio-compatible graph or mesh material.

In yet another embodiment, the present invention relates to an anchoring device for anchoring a sensor in a bodily vessel, which comprises a stent-like structure adapted to be secured in the bodily vessel, and a sensor operable to measure a physiologic parameter value in the bodily vessel. In this particular embodiment, the sensor can be attached to the stent-like structure so that the sensor is secured near the middle of the bodily vessel. For example, in one embodiment, the sensor can be attached to a leading edge of the stent-like structure using one or more connection structures, and in another embodiment, the sensor can be attached to a trailing edge of the stent-like structure using one or more connection structures.

In yet another embodiment, the present invention relates to an anchoring device for anchoring a sensor in a bodily vessel, which comprises a vena cava filter structure adapted to be, secured in the bodily vessel, and a sensor operable to measure a physiologic parameter value in the bodily vessel. In this particular embodiment, the sensor is attached to the vena cava filter structure. The vena cava filter structure can be any suitable vena cave filter currently known or later developed. For example, the filter can be an LGM filter, a Gunther tulipe filter, an Antheor filter, a DIL filter, a Keeper filter, a FCP2002 filter, a Mobin-Uddin filter, a Kimray-Greenfield filter, a Simon nitinol filter, a titanium Greenfield filter, a Bird's Nest filter, or the like.

In still another embodiment, the present invention relates to an anchoring device for anchoring a sensor in a bodily vessel, which comprises an anchoring structure adapted to be secured in the bodily vessel, and a sensor operable to measure a physiologic parameter value in the bodily vessel. In this particular embodiment, the anchoring structure comprises a plurality of extensions, which can be configured so that they will cause the anchoring structure to lodge in the bodily vessel. The sensor, in turn, is connected to the anchoring structure.

Yet another embodiment relates to a system including a vena cava filter anchorable at a position in a bodily vessel and one or more tethers each having a proximal end attached to the vena cava filter and a distal end attached to the IMD.

Another embodiment of an anchoring system includes a stent having one or more sleeves integrated in a wall of the stent, and the IMD includes one or more outwardly extending fins. The one or more fins can slide into and be held by the sleeves. In one embodiment, fibrosis can occur whereby tissue of the bodily vessel grows over the one or more fins to thereby secure the IMD to a wall of the bodily vessel. In this embodiment, the stent can be bio-absorbable.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

In the Figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates a sensor anchoring device in accordance with one embodiment of the present invention;

FIG. 2 is a top view of a section of the sensor anchoring device of FIG. 1 in which a sensor is placed;

FIG. 3 is a side view of the sensor anchoring device section and sensor illustrated in FIG. 2;

FIG. 4 is a cross-sectional view of one embodiment of a sensor anchoring device positioned within a bodily cavity;

FIG. 5 is a cross-section view of another embodiment of a sensor anchoring device positioned within a bodily cavity;

FIG. 6 is a view of one embodiment of a sensor device that can be anchored in a bodily cavity in accordance with one embodiment of the invention;

FIG. 7 is a cross-section view showing the sensor device of FIG. 6 being held in place in a bodily cavity by another embodiment of a sensor anchoring device;

FIG. 8 is an axial view showing the sensor device of FIG. 6 being held in place in a bodily cavity in accordance with one embodiment of an anchoring device;

FIG. 9 is a view of another embodiment of a sensor anchoring device;

FIGS. 10-12 are cross-section views of yet other embodiments of sensor anchoring devices positioned within bodily cavities;

FIG. 13 is a cross-sectional view of a heart showing the septal walls;

FIGS. 14a-14e are diagrams illustrating one embodiment of a method for anchoring a sensor within the septal wall of the heart; and

FIG. 15 is a flow diagram illustrating delivering, positioning, and anchoring a plug-like structure into a pre-anchoring slit according to one embodiment of the present invention;

FIG. 16 is a flow diagram illustrating an exemplary algorithm for controllably positioning and anchoring an IMD at a desired location.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Embodiments of the present invention generally relate to medical devices that can be implanted in a patient's body, and more particularly, to apparatus and methods for delivering, positioning, and anchoring implantable medical devices (IMDs) within a patient.

As discussed herein, various embodiments relate to systems and methods for obtaining physiologic parameter values and/or to provide therapeutic functions. For example, sensors or transducers can be placed in the body for monitoring a variety of properties, such as temperature, blood pressure, strain, fluid flow, chemical properties, electrical properties, magnetic properties within the body, bladder pressure, and the like. Exemplary types of sensors include, but are not limited to, thermometers, strain gauges, accelerometers, microphones, and the like. In addition, medical devices can be implanted that perform one or more therapeutic functions, such as drug delivery, cardiac pacing, defibrillation, electrical stimulation, and the like.

As mentioned, the physiologic parameter values obtained can be any physiologic measurement. Of course, as one skilled in the art will appreciate, measuring internal blood pressure (e.g., pulmonary artery pressures) is of particular interest for many clinical and therapeutic applications, and various embodiments are well suited to obtain blood pressure values. While embodiments discussed herein relate to sensors for measuring blood pressure, one skilled in the art will appreciate that the present invention is not limited to blood pressure measurements. More specifically, embodiments are not limited to IMDs capable of only performing sensing functions. As previously mentioned, various types of therapeutic functions are possible using IMDs. As such, embodiments are equally applicable to IMDs that are capable of sensing various physiological parameters, performing therapeutic functions, combinations of both sensing and therapeutic functionality, and any other function, or combination of functions related to medical treatment.

Also, as one skilled in the art will appreciate, blood pressure can be obtained from a number of different locations, such as the pulmonary artery, the aorta, anywhere in the circulatory system such as the venous system, or many locations distal from the heart. Different symptoms and/or diseases can be determined by measuring pressure at different locations. For example, the onset of pulmonary edema and hypertension can be detected by measuring pressure in the pulmonary artery. Similarly, arterial hypertension can be detected by measuring pressure in the aorta, and peripheral edema can be detected by measuring pressure in body extremities, such as the legs. Thus, as stated before, embodiments of the present invention are not limited to detecting pressure at any particular location. However, for ease of discussion, some of the anchoring methods and systems will described with reference to placing sensors in the pulmonary artery. As one skilled in the art will appreciate, however, the present invention is not limited to these particular embodiments.

The present application also is related to the following four co-pending patent applications: U.S. patent application Ser. No. 10/943,626 entitled “SYSTEMS AND METHODS FOR DERIVING RELATIVE PHYSIOLOGIC PARAMETERS,” and filed by Scott T. Mazar et al. (Attorney Docket No. 303437); U.S. patent application Ser. No. 10/943,269 entitled “SYSTEMS AND METHODS FOR DERIVING RELATIVE PHYSIOLOGIC PARAMETERS USING AN EXTERNAL COMPUTING DEVICE,” and filed by Jeffrey A. Von Arx et al. (Attorney Docket No. 306158); U.S. patent application Ser. No. 10/943,627 entitled “SYSTEMS AND METHODS FOR DERIVING RELATIVE PHYSIOLOGIC PARAMETERS USING A BACKEND COMPUTING SYSTEM,” and filed by Jeffrey A. Von Arx et al. (Attorney Docket No. 306664); and U.S. patent application Ser. No. 10/943,271 entitled “SYSTEMS AND METHODS FOR DERIVING RELATIVE PHYSIOLOGIC PARAMETERS USING AN IMPLANTED SENSOR DEVICE,” and filed by Abhi Chavan et al. (Attorney Docket No. 306663). All of the above-identified patent applications are incorporated by reference herein for all purposes. The above-identified patent applications will be referred to herein collectively as the “Physiologic Parameter Sensing Systems and Methods Patents.” In addition, the present application also is related to the following fifth co-pending patent application: U.S. patent application Ser. No. 11/168,282 entitled “ANCHOR FOR ELECTRODE DELIVERY SYSTEM,” and filed by Eric T. Johnson et al. (Attorney Docket No. 310008).

Brief definitions of terms and/or phrases used throughout this application are given below.

The phrases “in one embodiment,” “according to one embodiment,” and the like, generally mean the particular feature, structure, or characteristic following the phrase is included in at least one embodiment of the present invention, and may be included in more than one embodiment of the present invention. Importantly, such phrases do not necessarily refer to the same embodiment.

If the specification states a component or feature “may”, “can”, “could”, or “might” be included or have a characteristic, that particular component or feature is not required to be included or have the characteristic.

The term implantable medical device (IMD) includes devices capable of performing sensing, therapeutic functions, combinations of sensing and therapy, and the like. For purposes of illustration, and not limitation, various embodiments are described with respect to a sensor instead of the broader term IMD. In these cases, embodiments using sensors to illustrate the methods and systems apply equally to any IMD whether capable of sensing functionality or not.

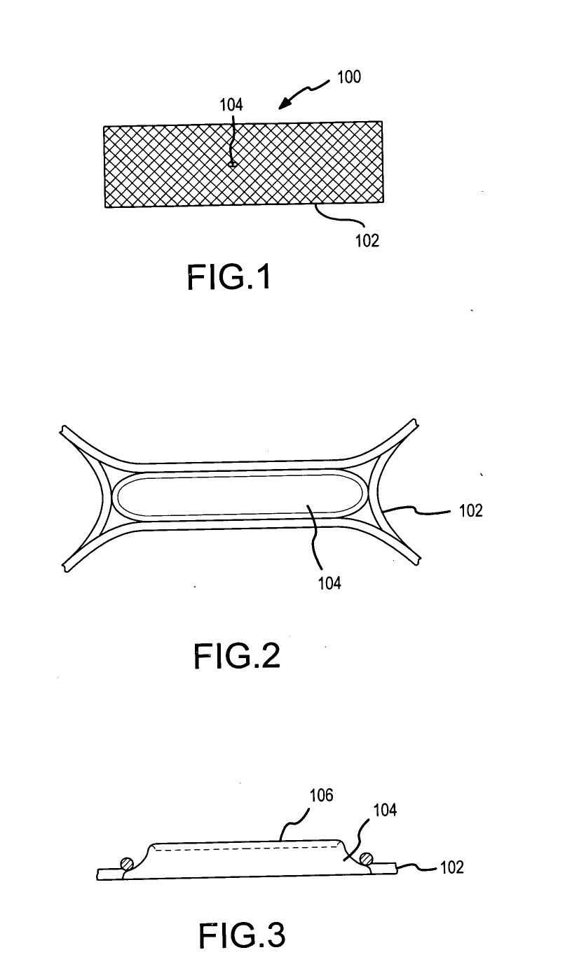

FIG. 1 shows one embodiment of a physiologic sensor anchoring system 100. In accordance with the illustrated embodiment, anchoring system 100 comprises a stent-like structure 102 carrying a physiologic parameter sensor 104 (e.g., pressure sensor). The stent-like structure generally has a tubular shape like a stent, and is adapted to carry the sensor 104 into a bodily vessel. In this particular embodiment, the physiologic parameter sensor 104 is embedded in a mesh structure of the stent-like structure 102, as is illustrated in a close-up view in FIG. 2.

The sensor 104 may be secured to and carried by the stent-like structure 102 in any number of ways. For example, as illustrated in FIG. 3, sensor 104 can rest in a recessed diaphragm 106 positioned in the stent 102. In alternative embodiments, sensor 104 can be secured within the stent using other securing mechanisms, such as adhesives, welding techniques, or the like. In addition, sensor 104 is configured to communicate with implantable medical devices (IMDs), such as cardiac rhythm management device, and/or devices outside of a patient body. A more complete description of sensors, sensor configurations, and communication systems and methods are discussed in more detail in the Physiologic Parameter Sensing Systems and Methods Patents incorporated by reference above.

In other embodiments, anchoring system 100 may be used for the placement of IMDs with therapeutic functions such as actuating devices. For example, common actuators include, but are not limited to, an ultrasound sensor and a drug delivery pod. In some embodiments, anchoring system 100 may be used to place a plurality of sensors, actuators, or a combination of sensors and actuators. Placement of multiple sensors and/or actuating devices throughout the body can allow for a more comprehensive therapeutic and diagnostic system, but multiple sensors and/or actuating devices are not required.

By using a stent-like anchoring structure, a sensor or any IMD can be anchored and secured in any part of the vascular system. In one particular embodiment, the stent-structure can be a balloon expandable stent, which can be placed in the vascular system using known catheterization techniques. For example, in one embodiment, the stent-structure can be positioned and secured in the pulmonary artery using techniques similar to a Swan Ganz technique, or other similar catheterization techniques. In this particular embodiment, when the stent-like anchoring mechanism 102 is expanded, sensor 104 will be place next to, or in close proximity to the vessel wall, allowing the sensor to obtain measurements from next to the vessel wall, which can be beneficial in many situations. As one skilled in the art will appreciate, for anchoring sensors in large cavities and/or arteries, stent-like anchoring mechanism 102 may be larger than a traditional stent device. However, the device configuration can be similar.

A balloon deployable stent can be made of stainless steel, cobalt chromium, nitinol, and the like. The material composition of the stent may be determined based on a variety of factors. For example, a stent placed in an artery in a patient's neck typically has a shape-memory because the stent may be deformed by exogenous pressures. In contrast, a stent positioned in the heart will have the protection of the patient's rib cage to help protect the stent from outside forces. Thus, it is not as important for a stent that is positioned in the heart to be made of a memory retaining material.

The stent is typically located on the outside of the balloon. As such, while inflating the balloon the stent expands. In many instances, it is desirable to activate and test the sensor during the placement, or positioning, phase. However, one potential problem with the balloon expandable stent approach is that while the balloon is inflated, the blood flow through the artery may be reduced or completely blocked. Hence, the sensor may not be able to provide an accurate measurement during placement. In addition, if the procedure is complicated, positioning of the sensor or actuator may take more time than the patient can safely be with reduced blood flow, or without blood flow entirely, in that area.

The balloon composed of a semi-permeable or permeable membrane. For example, the balloon may have holes, or paths, which allow the blood to flow. Another possible solution is for the balloon to be in a shape, such as a cloverleaf shape, that provides pockets through which blood can continue to flow while the balloon is inflated. A cloverleaf shape will not completely block the artery, as blood will be able to flow between the pedals of the clover shaped balloon. These techniques allow the sensor to be activated and tested during the positioning of the device, some benefits of which are discussed below.

In some embodiments, by using a stent-like anchoring structure, a physician can perform two functions at once; i.e., use a stent to expand and support a vessel while placing a physiologic parameter sensor in a desired location. Also, using a stent-like structure can have additional benefits, such as, for example: (1) the stent structure, if coated with one or more drugs to minimize inflammation, can help inhibit the long term inflammation of artery or vessel tissue, which can occur when other anchoring techniques are used; (2) when using a self expanding stent, the sensor can be tested prior to anchoring, and if there are problems with the sensor, it can be retracted prior to deploying the stent-like anchoring device; (3) the controlled deployment of the stent-structure can prevent incorrect anchoring within the artery or vessel, which can lead to serious thrombolytic effects; and (4) the stent-like structure might assist in evoking a limited tissue growth response over the sensor anchor, thus holding the sensor in place (a further embodiment of this concept is discussed in more detail below.

In accordance with these embodiments of the invention, the specific type of stent and its anchoring location is not limited. For example, the stent-like structure can be made of titanium, stainless steel, nitinol, or some other suitable bio-compatible material, and the stent-like structure design is not limited to any particular configuration. Further, as discussed above, the stent-like structure can be place in any part of the vascular system, including but not limited to, any venous or aortic blood vessel, the pulmonary artery, blood vessels distal from the heart, or any cardiac separating or enclosing wall (e.g., the atrial septum). In addition, as discussed above, the sensor can be configured to measure any physiologic parameter value, including any physical, chemical or biologic property or parameter. Finally, in one embodiment, the stent-like structure and/or sensor can be coated with drugs or other materials, which can reduce thrombolytic or inflammatory effects, promote fibrosis, or the like.

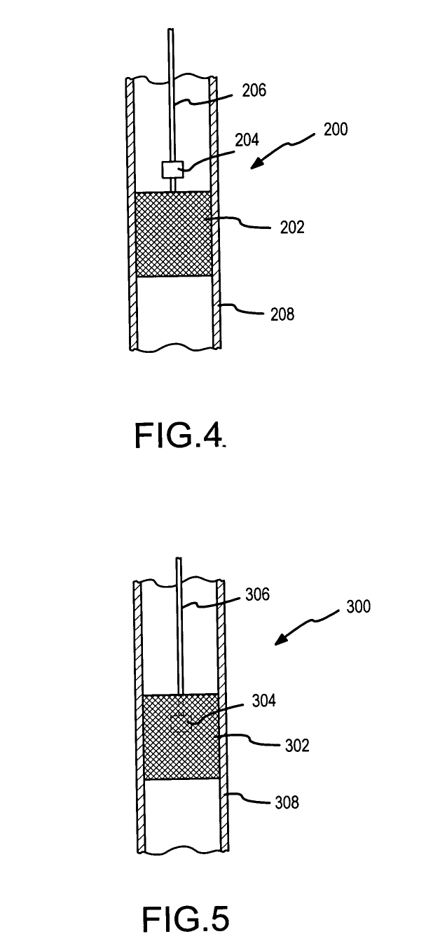

FIG. 4 illustrates another embodiment of a physiologic parameter sensor and anchoring system 200. In the embodiment illustrated in FIG. 4, system 200 comprises an anchoring device 202, a physiologic parameter sensor 204, and one or more leads 206 attached to sensor 204. In this particular embodiment, anchoring device 202 comprises a stent-like anchoring device, similar to the stent-like device discussed above. In FIG. 4, anchoring device 202 is shown expanded and anchored in a blood vessel 208. Again, as discussed above, vessel 208 can be any blood vessel within the body. In addition, anchoring device 202 is not limited to stent-like structure. Other anchoring devices, such as the devices discussed below, also can be used. Further, embodiments of the present invention are not limited to obtaining physiologic measurements within blood vessels.

In this particular embodiment, sensor 204 is attached or connected to lead 206, and lead 206 is further attached to anchoring device 202. Thus, the purpose of anchoring device 202 is to hold the sensor 204 and lead 206 configuration in a particular location in a vessel or other bodily cavity. As discussed in more detail in the Physiologic Parameter Sensing Systems and Methods Patents, lead 206 can facilitate communication between sensor 204 and an IMD, such as a cardiac rhythm management IMD. Lead 206 can carry sensor measurements from sensor 204 to the IMD, as well as therapy and/or other information from the IMD to the sensor 204. Further, lead 206 can be any suitable bio-compatible lead (e.g., silicone, polyurethane, etc.) currently known or later developed.

FIG. 5 shows yet another embodiment of a physiologic parameter sensor and anchoring system 300. In the embodiment illustrated in FIG. 5, system 300 also comprises an anchoring device 302, a physiologic parameter sensor 304, and one or more leads 306 attached to sensor 304 and/or anchoring device 302. According to various embodiments, the leads 306 may be a conductor, such as a braided cable. Examples of material from which the tether may be formed include, but are not limited to, MP35N, stainless steel, and other standard lead conductors. According to some embodiments, the diameters of the leads 306 typically range from 0.006 to 0.009 inches. In other embodiments, the diameters of the leads have a much larger range.

As with the embodiment illustrated in FIG. 4, anchoring device 302 comprises a stent-like anchoring device, but other anchoring devices can be used. In FIG. 5, anchoring device 302 is shown expanded and anchored in a blood vessel 308. Again, as discussed above, vessel 308 can be any blood vessel within the body, or any other bodily cavity, and embodiments of the present invention are not limited to obtaining physiologic measurements within blood vessels.

In this particular embodiment, sensor 304 is connected to anchoring device 302. Lead 306 is attached to sensor 304, and can be configured to communicate information to/from an IMD (e.g., a cardiac rhythm management IMD), as discussed in more detail in the Physiologic Parameter Sensing Systems and Methods Patents referenced above. For example, lead 306 can carry sensor measurements from sensor 304 to the IMD, as well as therapy and/or other information from the IMD to the sensor. Thus, one function of anchoring device 302 is to hold the sensor 304 in a particular location in a vessel or other bodily cavity, and one function of lead 304 is to facilitate communication with the IMD.

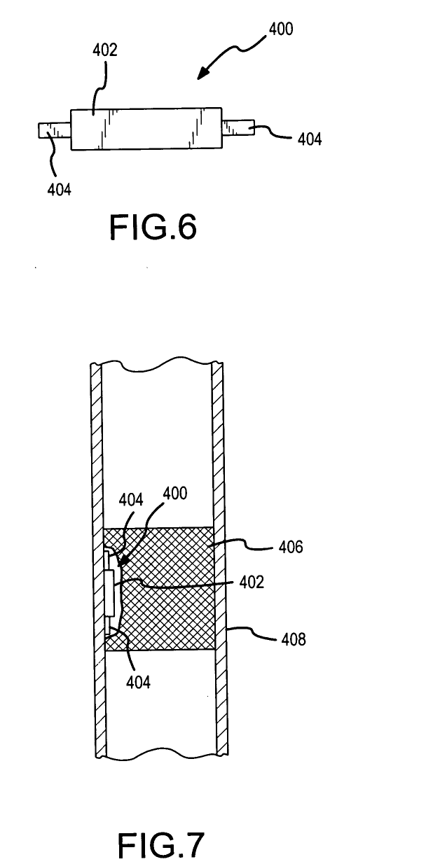

FIG. 6 illustrates one embodiment of a sensor device 400 that can be positioned and anchored within a bodily cavity, such as a blood vessel, or the like. In the embodiment illustrated in FIG. 6, sensor device 400 comprises a sensing mechanism (e.g., pressure sensor, circuitry, etc.) housed in a casing 402 that includes one or more fins or extensions 404 that can facilitate the anchoring of sensor device 400 in a bodily vessel. In addition to fins 404, the sensor 400 may have a Dacron skirt (not shown) that promotes fibrous ingrowth/overgrowth. In one embodiment, the skirt is similar to those used on myocardial leads. By the time the stent bio-absorbs, such a skirt will have securely grown to the wall of the vessel. The Dacron skirt can be positioned on the bottom of the sensor 400, but can also extend beyond the dimensions of the sensor 400.

According to some embodiments, the extension beyond the dimensions of the sensor 400 is similar to the configuration in epicardial (EPI) leads. As shown in FIG. 7, sensor device 400 can be positioned a within the bodily vessel (e.g., blood vessel 408 in FIG. 7), and initially anchored or held in place using an expandable stent-like device 406. As discussed above, stent-like device 406 can be any suitable stent device or other anchoring device currently known or later developed. In this particular embodiment, however, stent-like device 406 is bio-absorbable, and thus, will dissolve within a given time period (e.g., about 6-8 months).

In accordance with this particular embodiment, and as illustrated in FIG. 7, sensor device 400 is connected to anchoring device 406, so that sensor device 400, and in particular, the one or more fins 404, are positioned near the wall of vessel 408. The device 400 may be connected to the anchoring device 406 by a tether, a mold, dissolvable sutures, and the like. In any event, by placing the fins or extensions 404 near the vessel wall, tissue from the vessel will fibrose or grow over the fins 404, securing the sensor device 400 in the vessel. As one skilled in the art will appreciate, it may take time for fibrous tissue to form over extensions 404. As such, a relatively slow dissolving bio-absorbable anchoring device 406 is typically used to initially secure sensor device 400 in place. As one skilled in the art will appreciate, the vessel tissue typically will fibrose over extensions 404 within a period between about 3 months and 6 months, which is typically before anchor device 406 will completely dissolve.

With regard to embodiments that include outwardly extending fins 404, the stent-like structure 406 may include sleeves (not shown) formed on a wall of the stent-like structure 406 and configured for receiving and holding the fins 404. Thus, the sensor device 400 can be attached to the stent-like structure 406 by sliding the fins 404 into corresponding sleeves of the stent-like structure 406. The sleeves may be configured to allow for tissue fibrosis, thereby enabling gradual tissue growth over the fins 404 to secure the sensor device 400 to a wall of the bodily vessel 408.

In one embodiment, sensor device 400, including extensions 404 are formed from a bio-compatible material, such as stainless steel, titanium, nitinol, or some other bio-compatible material. In some embodiments, sensor casing 402 and extensions 404 are formed of the same material. In other embodiments, sensor casing 402 and extensions can be formed of different materials. In yet other embodiments, extensions 404 can comprise dacron, nylon or other bio-compatible graphs or patches, making it easier for tissue to adhere thereto. As one skilled in the art will appreciate, any number of extension 404 can be used, and extensions 404 can be any suitable size, shape and/or material. Thus, embodiments of the present invention are not limited to any particular material or extension 404 configuration illustrated and/or described herein. Further, in still other embodiments, sensor device 400 can be coated with one or more drugs that might help reduce inflammation and/or encourage or facilitate tissue fibrosis. Such drugs are currently known in the art.

In some embodiments, a fabric, such as Gore-Tex® (gore), may be placed between the stent and the sensor or actuator. The placement of this fabric facilitates in keeping the tissue from attaching to the sensor itself and only allows the tissue to grow around the stent. As such, the sensor, actuator, or some part of the circuitry such as the battery, may be detached, removed or replaced during a surgical procedure at a later time. For example, in FIG. 1 the sensor or actuator 102 may be removed, replaced, and reattached to anchoring mechanism 102 with a new sensor or actuator. In some embodiments, gore may also be used to cover both sides of the stent. In these embodiments, the stent is sandwiched between two layers of gore and the physical expansion of the stent holds the device in place, even with the gore sheets on either side. However, since tissue can not grow through the stent due to the gore, the entire stent may be more easily removed at a later time.

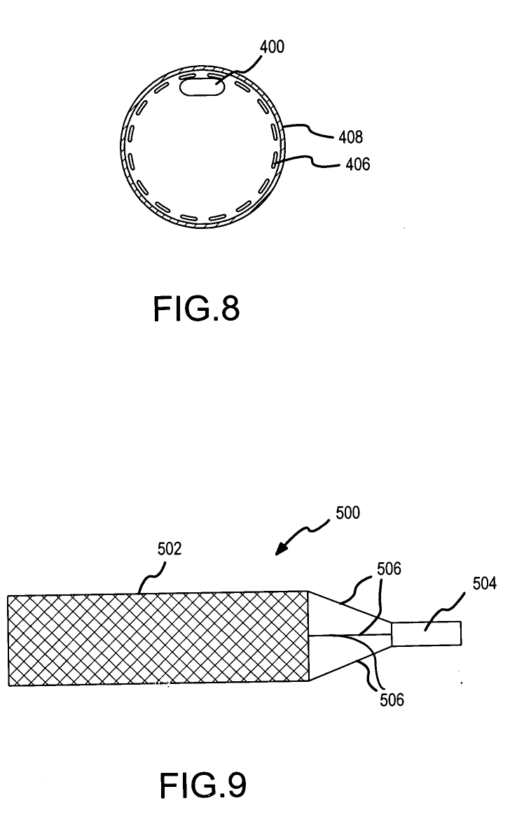

One embodiment, as illustrated in FIG. 7, has a sensor device 400 placed within the anchoring device 406. FIG. 8 shows an axial view of this embodiment. However, the anchoring device 406 may be placed on one side of the sensor device 400. Or, an anchoring device may be attached to both sides of the sensor or actuator's extensions or fins 404. This type of dual attachment of the sensor device 400 to one or more anchoring devices 406 may help facilitate more accurate final positioning of the sensor as both sides of the device may be anchored in place before the tissue grows around the device.

FIG. 9 shows yet another embodiment of an IMD anchoring system 500. In this particular embodiment, anchoring system 500 comprises an anchoring device 502, a sensor 504, and one or more connection structures 506 for connecting sensor 504 to anchoring device 502. In this particular embodiment, connection structures 506 are configured to secure sensor 504 so that the sensor will reside near the middle of a blood vessel. By placing the sensor 504 near the middle of the vessel, the sensor 504 will reside in the predominant blood flow that occurs in the middle of the vessel, avoiding edge effects, such as slower blood flow, dead zones, and perhaps clotting issues.

In one embodiment, anchoring device 502 can include a stent-like structure, as discussed above. Further, connection structures 506 can comprise any structural configuration that will secure sensor 504 in a desired location. For example, connection structures 506 can comprise one or more strut-type structures configured to hold sensor 504 in front of, or in back of anchoring device 502. In this particular embodiment, the strut-type structures can be made of the same material as the stent-like structure 502, or other materials can be used. Further, instead of securing sensor 504 in front of, or in back of anchoring device 502, connection structures can be used to secure sensor 504 within anchoring device 502, but still near the middle of the vessel. In addition, as discussed above, sensor 504 can be configured to communicate with implantable medical devices (IMDs), such as cardiac rhythm management device, and/or devices outside of a patient body. A more complete description of sensors, sensor configurations, and communication systems and methods are discussed in more detail in the Physiologic Parameter Sensing Systems and Methods Patents incorporated by reference above.

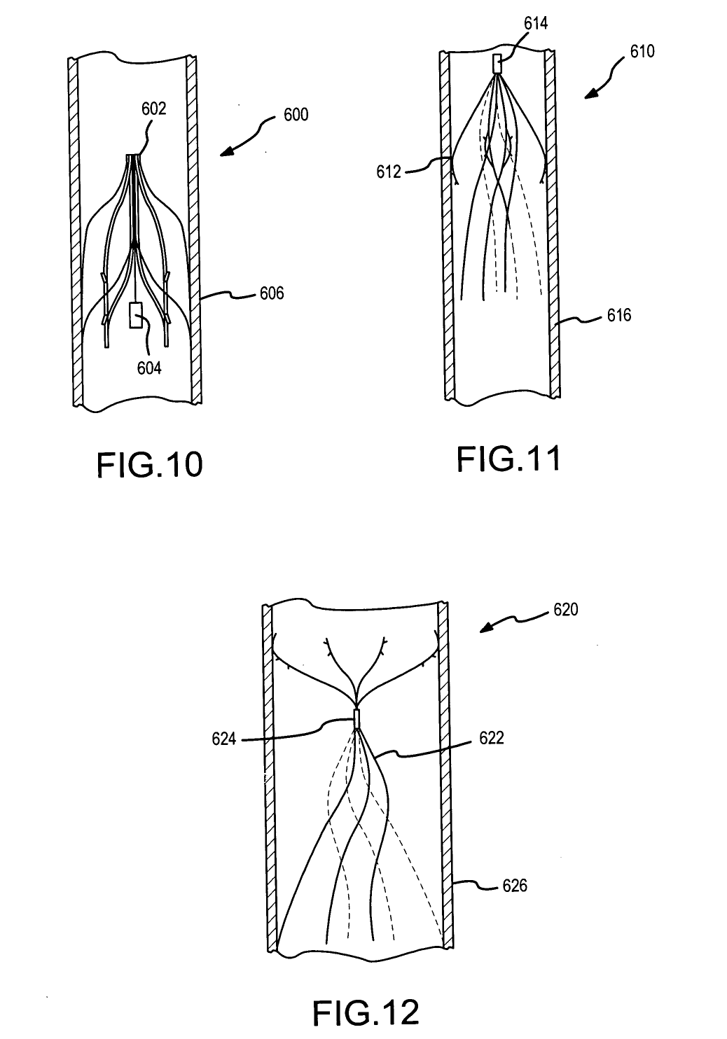

FIGS. 10-12 show additional embodiments of anchoring systems 600, 610, and 620. In these embodiments, anchoring structures 602, 612, and 622 can be used to secure sensors 604, 614, and 624 within a bodily vessel, such as a blood vessel. In some embodiments, the anchoring structure can be secured in place by surgical placement, and in other embodiments, the anchoring structure can be placed in a blood vessel, and then allowed to float or flow with the blood stream until the anchoring structure lodges in a suitable location to place the sensor.

In some embodiments (e.g., the embodiments illustrated in FIGS. 10-12), the anchoring structure can comprise a vena cava (“IVC”) filter device having a sensor attached to it. For example, as illustrated in FIG. 10, a sensor 604 can be connected to the IVC filter using a rigid or non-rigid tether connection. In other embodiments, such as the embodiments illustrated in FIGS. 11 and 12, sensors 614 and 624 can be incorporated into the structure of the IVC filter. In some embodiments, the sensor can be placed so that it is approximately near the center of the vessel to take advantage of the center flow of the vessel, and in other embodiments, the sensor can be configured so that it is secured near the wall of the vessel. Further, any suitable IVC filter device can be used. Examples of suitable IVC filters include, but are not limited to, an LGM filter, a Gunther tulipe filter, an Antheor filter, a DIL filter, a Keeper filter, a FCP2002 filter, a Mobin-Uddin filter, a Kimray-Greenfield filter, a Simon nitinol filter, a titanium Greenfield filter, a Bird's Nest filter, or any other suitable IVC filter device. Further, in other embodiments, the anchoring structures may not be IVC filters, but may comprise structures having legs or extensions for securing a sensor within a vessel. In these embodiments, the legs or extensions can be configured to lodge in the vessel in a manner similar to the IVC filters, thus securing the sensor in place.

In one embodiment, the anchoring structures are designed to be secured in the pulmonary artery, which branches and tapers as it flows toward the lungs. In this particular embodiment, the anchoring structure can be placed in the pulmonary artery, and then allowed to flow with blood stream until the anchoring structure lodges in a desired location. Once secured, the sensor can collect the desired data measurements. As one skilled in the art will appreciate, the size of the anchoring structure can control the location in which it will lodge. Also, as one skilled in the art will appreciate, the anchoring structure can be placed in other blood vessels, as well. Thus, embodiments of the present invention are not limited to use in the pulmonary artery.

A discussed above, sensors 604, 614 and 624 can be configured to communicate with implantable medical devices (IMDs), such as cardiac rhythm management devices, and/or devices outside of a patient's body. A more complete description of sensors, sensor configurations, and communication systems and methods are discussed in more detail in the Physiologic Parameter Sensing Systems and Methods Patents incorporated by reference above.

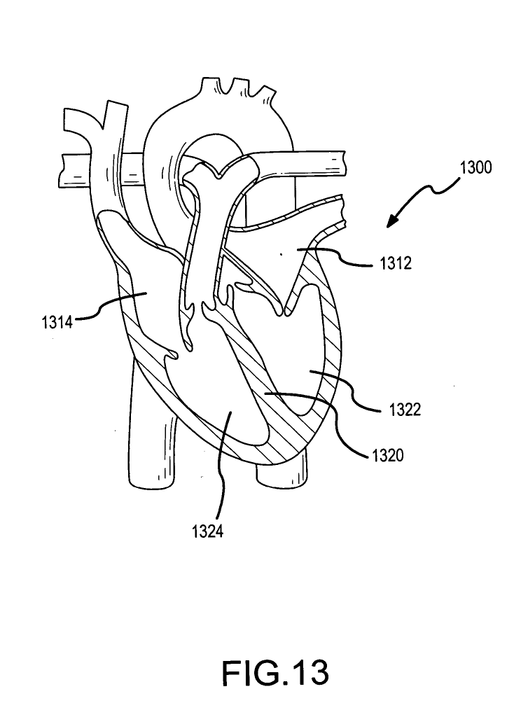

FIG. 13 shows a cross-sectional view of a heart 1300. As illustrated, heart 1300 includes an atrium septal wall (not shown) separating left atrium 1312 from right atrium 1314, and a ventricular septal wall 1320 separating left ventricle 1322 from right ventricle 1324.

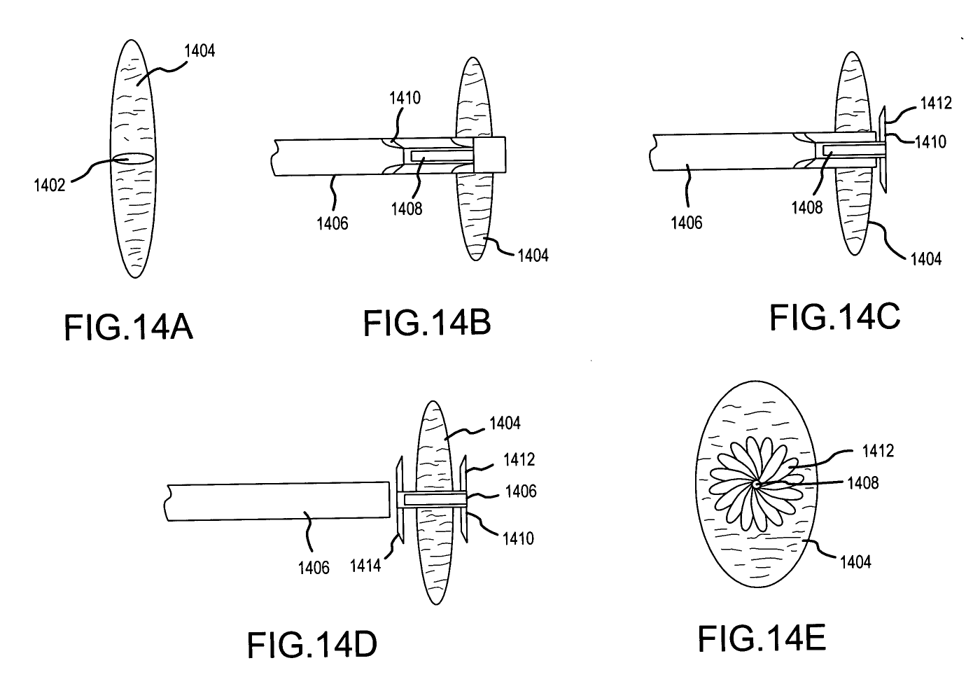

In accordance with another embodiment of the invention, a sensor anchoring device can be embedded in a separating or enclosing wall of the heart, for example, atrium septal wall or ventricular septal wall 1320. In FIGS. 14a-14e, one method of inserting a sensor anchoring device in accordance with this embodiment is shown. In this particular embodiment, a sensor 1408 can be embedded inside or attached to a plug-like anchoring structure, which then can be placed in any cardiac separating or enclosing wall 1404 (e.g., the septal wall). In accordance with this particular embodiment, a physician may be able to perform two functions at once: (1) fill a preexisting hole or slit in a cardiac separating wall in order to prevent blood from crossing from one side to another; and (2) use the plug as an enclosure for the placement of a physiologic parameter sensor. In other embodiments, a physician may create a hole or slit to place a sensor, and the plug-like anchoring structure can be used to place the sensor and plug and/or seal the slit or hole.

FIGS. 14a-14e illustrate one embodiment of a method for anchoring a sensor in a cardiac separating wall, such as the septal wall. FIG. 14a illustrates a cardiac separating wall (e.g., septal wall) 1404 with a hole or slit 1402 for placing an anchoring structure with sensor. As illustrated in FIG. 14b a physiologic parameter sensor 1408 embedded in or attached to a plug-like anchoring structure 1410 can be inserted into a pre-anchoring slit 1402 (either a nature hole or a surgically created hole or slit) using, for example, a guide catheter 1406. In this embodiment, the guide catheter has the anchor/sensor assembly embedded in it. To place the plug-like anchor 1410 (with sensor 1408) in the desired location, the guide catheter 1406 is placed in the hole or slit 1402 (FIG. 14b). Then, the guide catheter 1410 is retracted, causing plug ends 1412 and 1414 of the anchor device 1410 to expand (FIGS. 14c and 14d). The plug ends 1412 and 1414 form a seal so that blood cannot flow through hole 1402 or around anchor structure 1410. FIG. 14e shows an end view of plug end 1412 of the anchoring device 1410. In one embodiment, the anchoring device can be a septal plug currently known in the art. In this embodiment, however, the septal plug is equipped with a sensor, as discussed.

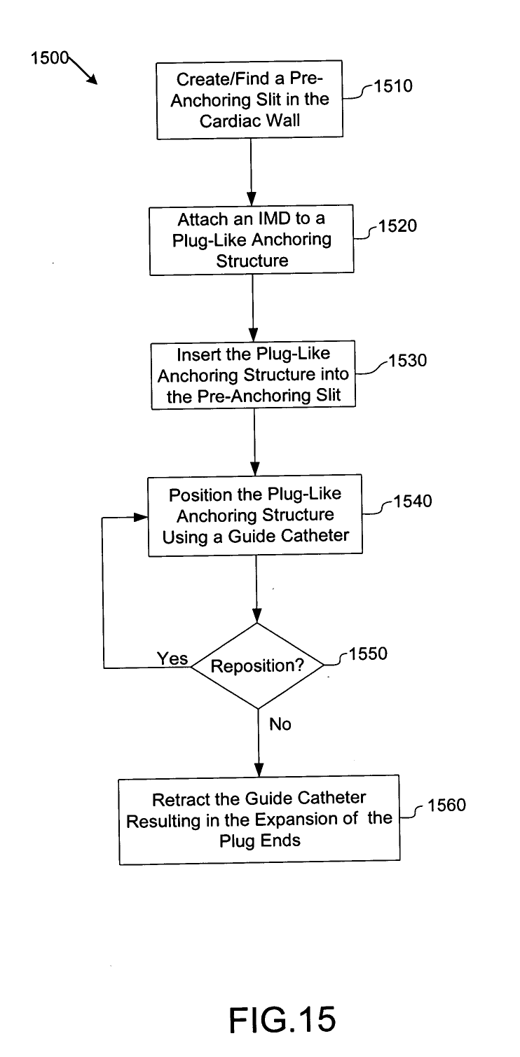

FIG. 15 is a flow diagram illustrating delivering, positioning, and anchoring a plug-like structure into a pre-anchoring slit according to one embodiment of the present invention. At block 1510, a pre-anchoring slit is located, or surgically created if one does not exist, in the cardiac wall. An IMD is attached to a plug-like anchoring structure at step 1520. Then, the plug-like anchoring structure is inserted into the pre-anchoring slit at step 1530. At step 1540, using a guide catheter, the plug-like anchoring structure is positioned and then repositioned, at step 1550, as necessary. Once the final placement of the plug-like anchoring structure has been achieved, the guide catheter is retracted, resulting in the expansion of the plug ends at step 1560.

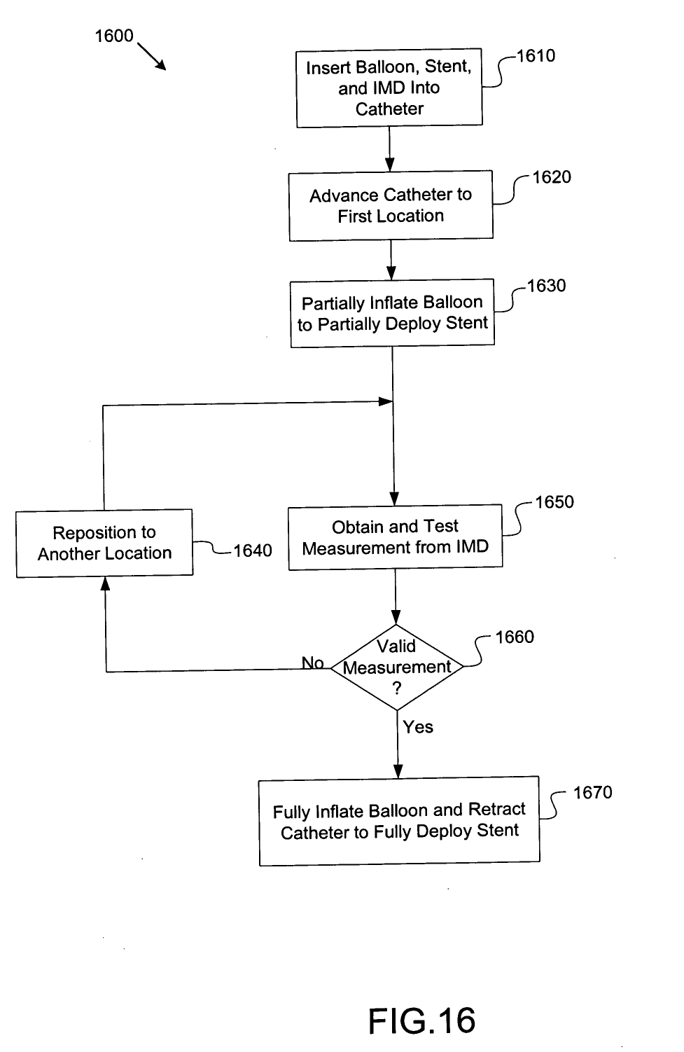

FIG. 16 is a flow diagram illustrating an exemplary algorithm 1600 for controllably positioning and anchoring an IMD at a location in a bodily vessel. At block 1610, a deflated balloon is inserted through a collapsed stent and an IMD is attached to the stent using, for example, one of the attachment methods described above. The stent with balloon and IMD are then inserted into a catheter.

At block 1620, the catheter is advanced into the bodily vessel to a first location. The first location is typically selected to be close to the desired location. At block 1630, the balloon is partially inflated, thereby partially expanding the stent. By partially inflating the balloon, the positioning can be controlled by enabling later repositioning, if desired. With the balloon partially inflated, one or more physiologic parameter measurements are obtained from the IMD (e.g., blood pressure, temperature, strain, motion, etc.) at block 1650. The measurements are tested for validity. Testing the measurements can involve determining whether numerical values are detected and that the values are reasonable.

At decision block 1660, it is determined whether the measurements are valid. If the measurements are not valid, block 1640 repositions the stent to another location by moving the catheter. After the stent is repositioned to the other location, block 1650 again obtains and tests measurements from the IMD. Repositioning can continue until block 1660 determines that the measurements are valid. If the measurements are valid, the balloon is fully inflated at block 1670 at the current location. By fully inflating the balloon, the stent if fully expanded. The fully expanded stent frictionally engages with walls of the bodily vessel to secure the stent within the bodily vessel.

As discussed, FIG. 16 illustrates a process for positioning a sensor using a balloon-deployable stent. A different embodiment could include self-expanding stent that carries the sensor. In this embodiment, the self-expanding stent can be partially deployed and tested prior to full deployment. If test measurements taken after partial deployment are not informative, invalid, or for any other reason, considered undesirable, or for any other reason (e.g., patient discomfort), the self-expanding stent can be moved to another location, tested, and so on. When valid test measurements are obtained at a location, the stent can be fully expanded at that location.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An anchoring system for anchoring an implantable medical device (IMD) in a bodily vessel of a patient, the anchoring system comprising:
an anchoring structure adapted to be secured in the bodily vessel;
an attachment structure integrated with the anchoring structure, the attachment structure enabling attachment of the IMD to the anchoring structure; and
a positioning mechanism operable to position the anchoring structure at a desired location in the bodily vessel.

2. An anchoring system as recited in claim 1 wherein the anchoring structure comprises a stent-like structure composed of mesh fabric forming the attachment structure, wherein the IMD is embedded in the mesh fabric.

3. An anchoring system as recited in claim 1 wherein the anchoring structure comprises a stent-like structure having a recessed diaphragm forming the attachment structure, wherein the IMD is positioned in a recess formed by the recessed diaphragm.

4. An anchoring system as recited in claim 1 wherein the positioning mechanism comprises a catheter, and wherein the anchoring structure comprises a stent that is balloon deployable from the catheter.

5. An anchoring system as recited in claim 4 wherein the stent is controllably deployable, whereby measurements obtained by the IMD can be tested while the stent is being deployed.

6. An anchoring system as recited in claim 1 wherein the anchoring structure comprises a stent, and the anchoring structure comprises sleeves integrated in a wall of the stent, wherein one or more fins extending from the IMD can be held by the sleeves.

7. An anchoring system as recited in claim 6 wherein fibrosis causes tissue of the bodily vessel to grow over the one or more fins, thereby securing the IMD to a wall of the bodily vessel.

8. An anchoring system as recited in claim 6 wherein the stent is bio-absorbable.

9. An anchoring system as recited in claim 1 wherein the attachment structure further comprises one or more leads, each lead having a proximal end attached to the anchoring structure and a distal end attached to another IMD.

10. An anchoring system as recited in claim 1, further comprising a lead communicably connected to the IMD, the lead being operable to communicate data from the IMD to another device implanted in the patient.

11. An anchoring system as recited in claim 1, wherein the IMD is operable to measure one or more physiologic parameters, the one or more physiologic parameters including at least one of blood pressure, motion, temperature, strain, and sound.

12. An anchoring device of claim 1, wherein the attachment structure holds the IMD substantially in a middle region of the bodily vessel.

13. A system for anchoring an implantable medical device (IMD) at a position in a bodily vessel, the system comprising:
a vena cava filter anchorable at the position in the bodily vessel;
one or more tethers each having a proximal end attached to the vena cava filter and a distal end attached to the IMD.

14. The anchoring device as recited in claim 13, wherein the vena cava filter is selected from the group of vena cava filters consisting of an LGM filter, a Gunther tulipe filter, an Antheor filter, a DIL filter, a Keeper filter, a FCP2002 filter, a Mobin-Uddin filter, a Kimray-Greenfield filter, a Simon nitinol filter, a titanium Greenfield filter, and a Bird's Nest filter.

15. A method for anchoring an implantable medical device in a bodily vessel, the IMD operable to measure a physiologic parameter within the bodily vessel, the method comprising:
attaching a first implantable medical device (IMD) to a stent-like structure, wherein attaching comprises securing at least a portion of the IMD in a structure integrated with the stent-like structure;
positioning the stent-like structure at a desired location within the bodily vessel; and
anchoring the stent-like structure at the desired location within the bodily vessel.

16. A method as recited in claim 15 wherein positioning comprises:
positioning the stent-like structure at a first location within the bodily vessel;
testing a measurement from the first IMD; and
based on the testing, repositioning the stent-like structure to a second location within the bodily vessel.

17. A method as recited in claim 15 wherein securing the first IMD comprises positioning the first IMD within a reservoir integrally formed in a wall of the stent-like structure.

18. A method as recited in claim 15 wherein securing the first IMD comprises lodging the first IMD in a mesh fabric forming the stent-like structure.

19. A method as recited in claim 15 wherein the attachment structure comprises one or more sleeves integrated with a wall of the stent-like structure, and wherein the first IMD comprises one or more fins extending outward from the IMD and configured to slide into the one or more sleeves.

20. A method as recited in claim 19 wherein the stent-like structure is permeable by bodily tissue and wherein the fins are configured to enable fibrosis of the bodily tissue to gradually attach the IMD to a wall of the bodily vessel.

21. A method as recited in claim 15 further comprising communicably connecting the first IMD to a second IMD via a lead, whereby measurements obtained by the first IMD can be communicated to the second IMD.

22. The method of claim 15, wherein the stent-like structure comprises a balloon deployable stent wrapped around a balloon, and wherein positioning comprises:
inserting the balloon deployable stent and balloon into a catheter;
advancing the catheter into the bodily vessel to position the stent and the first IMD at a first location within the bodily vessel;
partially inflating the balloon to cause the stent to partially expand;
testing a measurement from the IMD at the first location; and
based on the measurement, moving the catheter to position the stent at a second location while the stent is partially expanded.

23. A method as recited in claim 22 further comprising testing another measurement from the IMD at the second location, and wherein anchoring comprises:
based on the another measurement, completely inflating the balloon, thereby fully expanding the stent to create a frictional engagement of the stent with walls of the bodily vessel.

24. A method for anchoring an implantable medical device (IMD), comprising:
connecting a collapsible stent to an IMD operable to measure a physiologic parameter;
inserting a deflated balloon through the stent with the stent collapsed;
positioning the stent with the balloon and the IMD inside a bodily vessel;
while positioning the stent, obtaining measurements from the IMD;
at least partially inflating the balloon; and
selecting a location in the bodily vessel at which to secure the stent based on the obtained measurements.

25. A method as recited in claim 24 wherein the balloon is configured to allow fluid flow through the bodily vessel.

26. A method as recited in claim 25 wherein the balloon is a clover-leaf shape

27. A method as recited in claim 25 wherein the balloon is semi-permeable.
